(19) United States
(12) Patent Application Publication
(10) Pub. No.: US 2005/0049620 A1
(43) Pub. Date: Mar. 3, 2005

(54) Electric nose suction-washer

(76) Inventor: Hueydy Chang, Longtan Township (TW)

Correspondence Address: Hueydy Chang, P.O. Box No. 6-57, Junghe, Taipei, 235, TW

(21) Appl. No.: 10/651,974

(22) Filed: Sep. 2, 2003

(51) Int. Cl. A61F 9/00

(52) U.S. Cl. 606/162

(57) ABSTRACT

An improvement on the structure of electric nose suction-washer consists of a body for grip and for casing a motor mechanism. At the tip of the body are the suctioning and washing units of the motor mechanism for sucking nasal fluid or washing the nasal cavity. Therein, the body is cylindrical at a suitable place of which is a droplet shaped protrusion for easy gripping. Inside a bottom of the body encases the battery case where a spring plate exists for ease of replacing batteries and for forming power connection to the motor mechanism. The inner wall of the battery case is a plurality of vertical ridges for positioning and fastening the battery case.

### BACKGROUND OF THE INVENTION

(a) Field of the Invention

The invention relates to an electric nose suction-washer, especially one that is easy to grip and change batteries.

(b) Description of the Prior Art

According to an electric nasal cavity washer or nasal fluid suctioner of the prior art as illustrated in FIG. 1, the inverted L-shaped body 1 has a motor mechanism of suctioning and washing units 11 at its tip for sucking nasal fluid and washing the nasal cavity. The body 1 of the prior art is smooth lacking a place for a firm grip so it is easy to fall out of hand when in use. Furthermore, the power supply comes from a rechargeable battery 14 of a battery case 12 connected to the body 1 by a guide wire 13. When in use the power goes through a transformer to drive the motor mechanism. However, without available power source to recharge, the prior art will be rendered inoperable and unable to substitute with alkaline batteries.

To enable a further understanding of the said objectives and the technological methods of the invention herein, the brief description of the drawings below is followed by the detailed description of the preferred embodiment.

### BRIEF DESCRIPTION OF THE DRAWINGS

FIG. 1 shows a perspective exploded view of the entire structural combination according to the prior art.

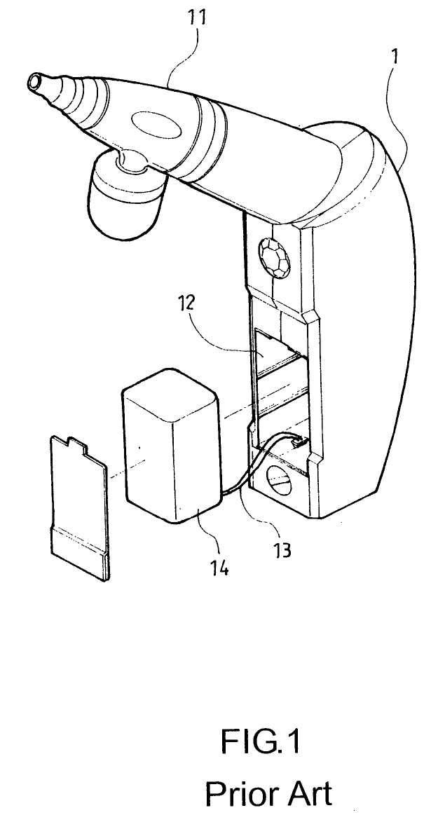

FIG. 2 shows a perspective view of the entire structural combination according to the invention.

FIG. 3 shows a perspective exploded view of the entire structural combination according to the invention.

FIG. 4 shows a partial cross-sectional and exploded view of the battery case according to the invention.

### SUMMARY OF INVENTION

The goal of the invention is to provide an improvement on the prior art of an electric nose suction-washer for easy gripping, operation and replacement of batteries.

The invention relates to an inverted L-shaped cylindrical body which houses the motor mechanism of suctioning and washing units at the tip for sucking nasal fluid or washing the nasal cavity. Therein, at a suitable place of an upper part of the body is a horizontal droplet shaped protrusion for easy gripping and preventing the invention from falling off the hand.

The invention also relates to a battery case at the bottom of the body. Alkaline (rechargeable) batteries can be placed into a battery box which can be inserted into the battery case. A spring plate connects the battery case to the motor mechanism to provide power. The replacement of batteries has become more convenient and less limited by available power source.

### DETAILED DESCRIPTION OF THE PREFERRED EMBODIMENTS

To better understand the characteristics and novelties of the invention, descriptions shall be given with the accompanying drawings hereunder.

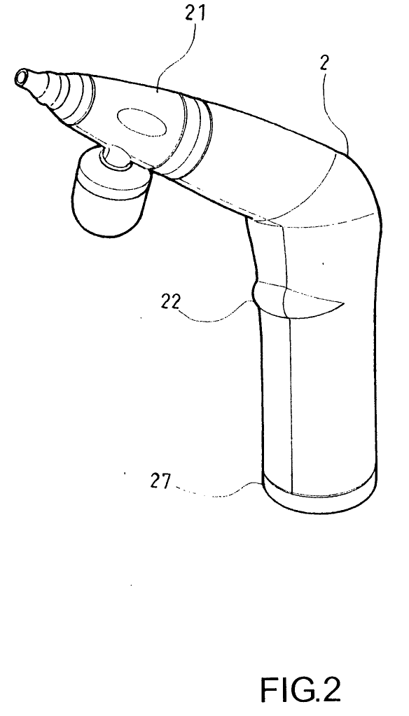

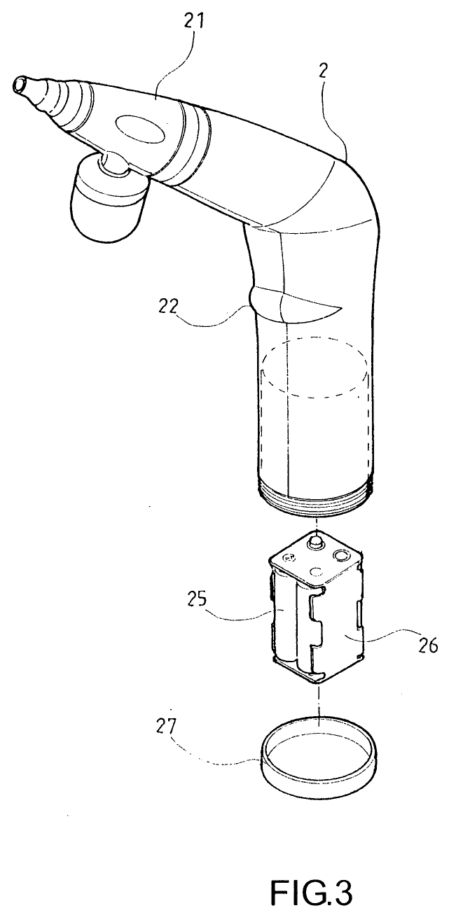

Referring to FIGS. 2 and 3, the invention showing an inverted L-shaped cylindrical body 2 inside which is a motor mechanism of washing (or suctioning) unit 21 at a tip of the body 2 for sucking nasal fluid or washing the nasal cavity. Therein, at a suitable place of an upper part of the body 2 is a horizontal droplet shaped protrusion 22 for easy operation, comfortable gripping of hand on the body 2 and for preventing the invention from falling off the hand.

At a bottom of the body 2 is a battery case 23. After alkaline batteries (or rechargeable batteries) 25 are placed into a battery box 26 which is then put into the battery case 23, a cap 27 can twist shut the battery case 23 at the bottom of battery case 23 by a twist groove 24.

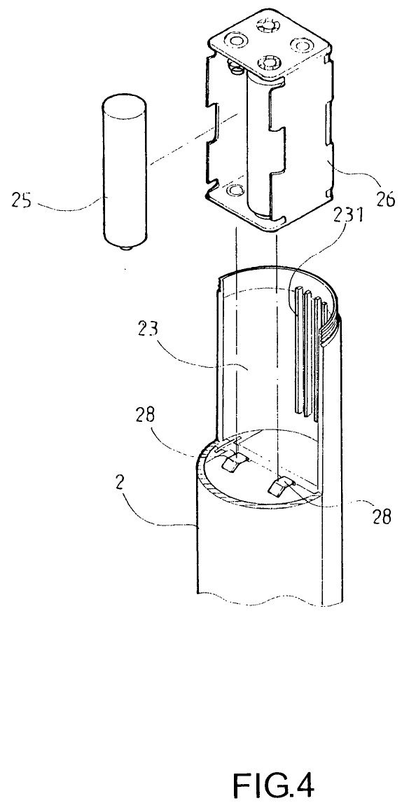

Referring to FIG. 4, inside the battery case 23 is a spring plate 28 connected to the motor mechanism. A plurality of vertical ridges 231 are in the inner wall of the battery case 23. A tip of the battery box 26 touches the spring plate 28 after inserting the battery box 26. Through the spring plate 28, voltage is passed to the motor mechanism. When the power runs out, the batteries 25 can be replaced immediately, thus improving the limitation of rechargeable batteries by the availability of power source. Furthermore, the invention operates on alkaline batteries so that replacing batteries has become more effortless. Also that the inner wall of the battery case has a plurality of vertical ridges so that the battery box is affixed to the proper position.

In conclusion, the invention—electric nose suction-washer—consisting of a protrusion on the body, a battery case with spring plate for a more convenient cleaning of the nose and replacement of batteries is of a practical design and new invention and thus this application of patent according to the rules and regulations.

It is of course to be understood that the embodiment described herein is merely illustrative of the principles of the invention and that a wide variety of modifications thereto may be effected by persons skilled in the art without departing from the spirit and scope of the invention as set forth in the claims.

1. An electric nose suction-washer comprising
an inverted L-shaped cylindrical body encasing a motor mechanism of suctioning and washing units at a tip thereof for sucking nasal fluid or washing nasal cavity, of wherein,
at a suitable place of an upper part of the inverted L-shaped cylindrical body is a horizontal droplet shaped protrusion for easy gripping, a battery case at a bottom of the inverted L-shaped cylindrical body connected by a spring plate to the motor mechanism to facilitate replacement of batteries, and in an inner wall of the battery case are vertical ridges for fastening and positioning of the battery box.
